(19) United States
(12) Patent Application Publication
(10) Pub. No.: US 2017/0290740 A1
(43) Pub. Date: Oct. 12, 2017

(54) COLONOSCOPY PREP BAG AND METHODS OF USE

(71) Applicant: Gavis Pharmaceuticals, Somerset, NJ (US)

(72) Inventor: Veerappan Subramanian, Somerset, NJ (US)

(73) Assignee: Gavis Pharmaceuticals, Somerset, NJ (US)

(21) Appl. No.: 15/056,551

(22) Filed: Feb. 29, 2016

Related U.S. Application Data
(60) Provisional application No. 62/121,629, filed on Feb. 27, 2015.

(51) Int. Cl. A61J 1/10; A61K 33/00; A61K 33/04; B65D 75/56; A61J 1/14; A61J 1/18; B65D 75/58; B65D 75/00; A61K 47/34; A61K 9/00

(52) U.S. Cl. CPC A61J 1/10; A61K 47/34; A61K 33/00; A61K 33/04; A61K 9/0095; A61K 9/0053; A61J 1/1418; A61J 1/18; A61J 1/1468; B65D 75/5883; B65D 75/008; B65D 75/566; A61J 1/1475

(57) ABSTRACT

The invention relates to a pharmaceutical composition of polyethylene glycol in solid powder form contained within a polymer bag having an opening. The invention also provides a method for administering a pharmaceutical composition of polyethylene glycol in solid powder form. The method includes providing a polymer bag containing the pharmaceutical composition of polyethylene glycol and adding a fluid to the bag. The polymer bag has a threaded spout and a cap threadably mountable to the spout. The threaded spout forms a passageway to an interior volume of the bag.

### CROSS-REFERENCE TO RELATED APPLICATION

The present application relates to, and claims priority to U.S. Provisional Patent Application Ser. No. 62/121,629 filed on Feb. 27, 2015, which is entitled “Colonoscopy Prep Bag and Methods of Use”. The content of the provisional application is incorporated herein in its entirety by reference.

### TECHNICAL FIELD OF THE INVENTION

The technical field of the invention relates to colonoscopy prep bags, colonoscopy prep bags containing the colon cleansing material, and methods of use of the colonoscopy prep bags to prepare for a colonoscopy procedure.

### BACKGROUND OF THE INVENTION

A colonoscopy is a medical procedure for examining the inner lining of the large intestine, typically to examine for ulcers, polyps, tumors or areas of inflammation or bleeding. To conduct a colonoscopy examination, a physician inserts an endoscope through the anus and advances the endoscope into the colon. The endoscope is a thin, flexible tube that typically contains a camera, and multiple channels for instrumentation, air, suction and light. During a typical procedure, the physician will use a light source and camera to search for polyps. Because of the potentially small size of a polyp, the inner lining of the intestine must be clean of all solid materials.

To ensure that the intestine is clean and free of solid materials a patient is advised to follow a cleansing regimen the day before the colonoscopy. The patient may be given a laxative preparation and large quantities of fluids to flush the colon of solid materials. The patient also or instead may be asked to perform a whole bowel irrigation by first taking a laxative in pill form and then drinking a solution of polyethylene glycol and electrolytes in a clear liquid. For example, the patient may mix a solid powder of polyethylene glycol with a clear sports drink that contains electrolytes. Once the polyethylene glycol has dissolved in the fluid, the patient then drinks the mixture. One method used to mix and administer the polyethylene glycol mixture is by supplying the solid powder mixture in a jug and adding water to the jug. Referring to FIGS. 1 and 2, as is known, a conventional reconstitution jug 10 is a container that has an opening or mouth 15, a top or lid 20, a handle 25, a generally round side wall 30 and a bottom wall 35. The mouth 15 has a threaded neck 40 onto which the lid 20 is threadably mounted. The side wall and bottom wall typically is relatively opaque.

A common fluid regimen for the whole bowel irrigation is to mix polyethylene glycol power in 64 fluid ounces of a clear sports drink in a conventional reconstitution jug. Approximately two hours after taking the laxative pills, the patient begins drinking the fluid at a rate of 8 ounces every fifteen to thirty minutes until the entire 64 ounces are consumed. Two such products available for this procedure are available from Novel Laboratories, Inc. that contain polyethylene glycol 3350, potassium chloride, sodium bicarbonate and sodium chloride or polyethylene glycol 3350, potassium chloride, sodium bicarbonate, sodium chloride and sodium sulfate anhydrous. These products are available in large, disposable plastic jugs that hold approximately four liters of fluid. Other products available for cleansing the colon in preparation for a colonoscopy are Prepopik®, which contains sodium picosulfate, magnesium oxide and anhydrous citric acid, and OsmoPrep®, which contains sodium phosphate monobasic monohydrate, USP, and sodium phosphate dibasic anhydrous, USP.

For the Novel Laboratories product described above, the product dispensed by the pharmacist to the patient preparing for the colonoscopy is a jug that contains in solid powder form: 236 grams of polyethylene glycol 3350, 22.74 grams of sodium sulfate (anhydrous), 6.74 grams of sodium bicarbonate, 5.86 grams of sodium chloride, and 2.97 grams of potassium chloride. A flavour packet is supplied separately with the jug and added to the solid mixture in the jug at the time of dispensing by the pharmacists. The jug is marked with a visual indication for filling to four liters of fluid. The instructions for the product state that the container should be filled to the four liter mark with water and shaken vigorously several times to ensure dissolution of the ingredients. The instructions note that warm water facilitates dissolution but the solution is more palatable if chilled before administration. The reconstituted solution should be kept refrigerated and used within 48 hours.

### SUMMARY OF THE INVENTION

In one general aspect the invention provides a pharmaceutical composition comprising a colonoscopic bowel preparation in solid powder form contained within a polymer bag having an opening.

Embodiments of the pharmaceutical composition may include one or more of the following features. For example, the pharmaceutical composition may include polyethylene glycol 3350, potassium chloride, sodium bicarbonate and sodium chloride. In another embodiment, the pharmaceutical composition may include polyethylene glycol 3350, potassium chloride, sodium bicarbonate, sodium chloride and sodium sulfate anhydrous. The pharmaceutical composition may include about 200 to 250 grams of polyethylene glycol 3350, 2.5 to 3.5 grams of potassium chloride, 6.5 to 7.5 grams of sodium bicarbonate and 5.5 to 6.5 grams of sodium chloride. The pharmaceutical composition may include about 236 grams of polyethylene glycol 3350, about 2.97 grams of potassium chloride, about 6.74 grams of sodium bicarbonate and about 5.86 grams of sodium chloride.

In one embodiment the polymer bag may be a high density polyethylene polymer. The polymer bag may be a clear polymer material. The polymer bag may include one or more fill markings on the bag. The polymer bag may have a volume of approximately 4 liters when filled with fluid.

The polymer bag may include at least one opening through the bag with the opening in the bag being defined by a continuous edge of the polymer bag forming a seal between an interior volume of the bag and the opening through the bag.

The opening in the bag may be formed by a threaded spout and a cap threadably mountable to the spout with the threaded spout forming a passageway to an interior volume of the bag. The bag may include a base, i.e., a base support.

In another general aspect, the invention provides a method for administering a pharmaceutical composition comprising a colonoscopic bowel preparation in solid powder form. The method includes providing or obtaining a polymer bag containing a pharmaceutical composition comprising the colonoscopic bowel preparation in solid powder form and adding a fluid to the bag. The polymer bag has a threaded spout and a cap threadably mountable to the spout. The threaded spout forms a passageway to an interior volume of the bag.

Embodiments of the method of administering the pharmaceutical composition may include one or more of the following features. For example, the method may further include mixing the colonoscopic bowel preparation in solid powder form with the fluid to dissolve the colonoscopic bowel preparation in solid powder form. Approximately four liters of fluid may be added to the bag. A first portion of the fluid may be added to the bag until a mark on a surface of the bag is reached, the bag shaken to mix the pharmaceutical composition with the fluid, and then a second portion of the fluid added to the bag.

The pharmaceutical composition may include polyethylene glycol 3350, potassium chloride, sodium bicarbonate, sodium chloride and, optionally, sodium sulfate anhydrous. The pharmaceutical composition may include about 200 to 250 grams of polyethylene glycol 3350, 2.5 to 3.5 grams of potassium chloride, 6.5 to 7.5 grams of sodium bicarbonate and 5.5 to 6.5 grams of sodium chloride.

The polymer bag may be formed from a clear polymer material. The polymer material may be a high density polyethylene.

In another general aspect, the invention provides a method for administering a pharmaceutical composition comprising a colonoscopic bowel preparation in solid powder form. The method includes prescribing a polymer bag containing a pharmaceutical composition comprising the colonoscopic bowel preparation in solid powder form configured for adding a fluid to the bag. The polymer bag has a threaded spout and a cap threadably mountable to the spout. The threaded spout forms a passageway to an interior volume of the bag.

Still other aspects and advantages of the invention will be apparent from the following detailed description of the invention. For example, in comparision to a conventional jug to contain the solid powder material that is mixed with a liquid, the bag will significantly reduce the amount of space required for shipping and storage. In one embodiment, the bag will take 8-12 times less space than a jug that is designed to contain the same volume of fluid. This space saving aspect of the invention provides cost, storage, shipping and ecological advantages. For example, the vehicles that transport the bag will be able to move 8-12 times more bags than jugs intended to contain the same volume. Similarly, a pharmacy will be able to store the same number of bags as jugs but do so in 8-12 times less space. And after use by the patient, the bag can be repurposed and reused to hold water for outdoor activities. These advantages are surprising and unexpected.

### DESCRIPTION OF THE DRAWINGS

FIG. 1 is a perspective view of a conventional reconstitution jug.

FIG. 2 is a side view of the mouth of the reconstitution jug of FIG. 1.

FIG. 3 is a side view of a reconstitution bag.

FIGS. 4a-f are a series of side views illustrating a process of forming the reconstitution bag of FIG. 3.

FIG. 5 is a side view of the reconstitution bag of FIG. 3 filled with a colonoscopic bowel preparation in solid powder form.

FIG. 6 is a perspective view of the reconstitution bag of FIG. 3 in an upright standing position.

FIGS. 7A and 7B are the front and back views of the reconstitution bag of FIG. 3.

FIGS. 8A and 8B are the left and right side views of the reconstitution bag of FIG. 3.

FIGS. 9A and 9B are the bottom and top views of the reconstitution bag of FIG. 3.

FIGS. 10A and 10B are perspective views of the tap and gland which is used in conjunction with the reconstitution bag of FIG. 3.

FIGS. 11A, 11B and 11C are the perspective and front views of a first child restraint cap to be used in conjunction with the reconstitution bag of FIG. 3.

FIGS. 12A and 12B are the perspective and side views of a second child restraint lock to be used in conjunction with the reconstitution bag of FIG. 3.

FIGS. 13A and 13B depict a second embodiment of the invention.

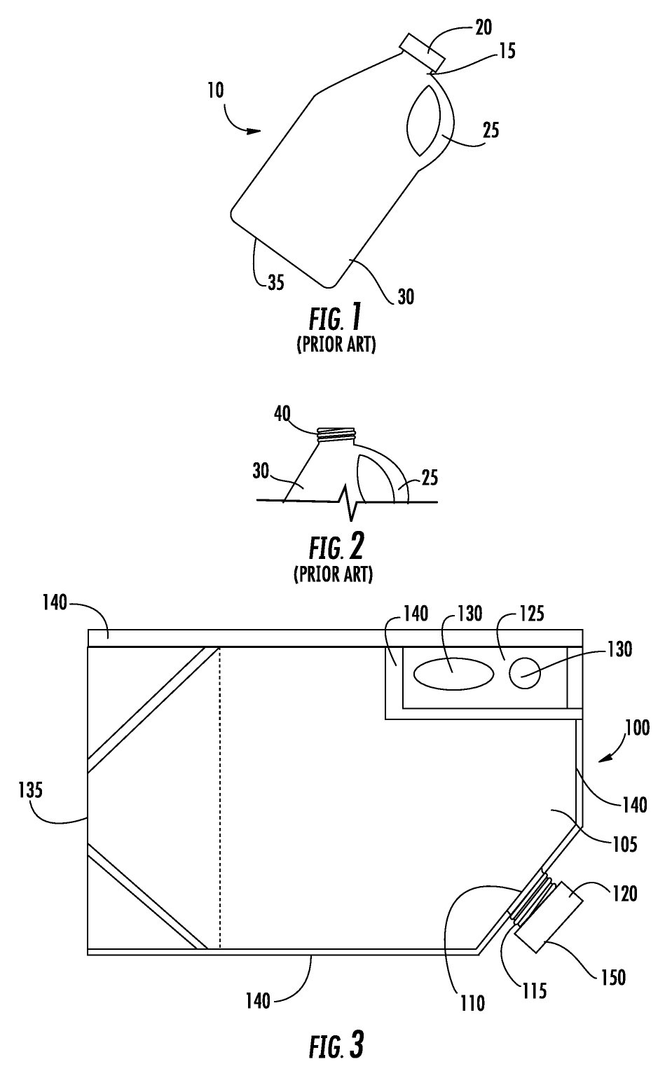

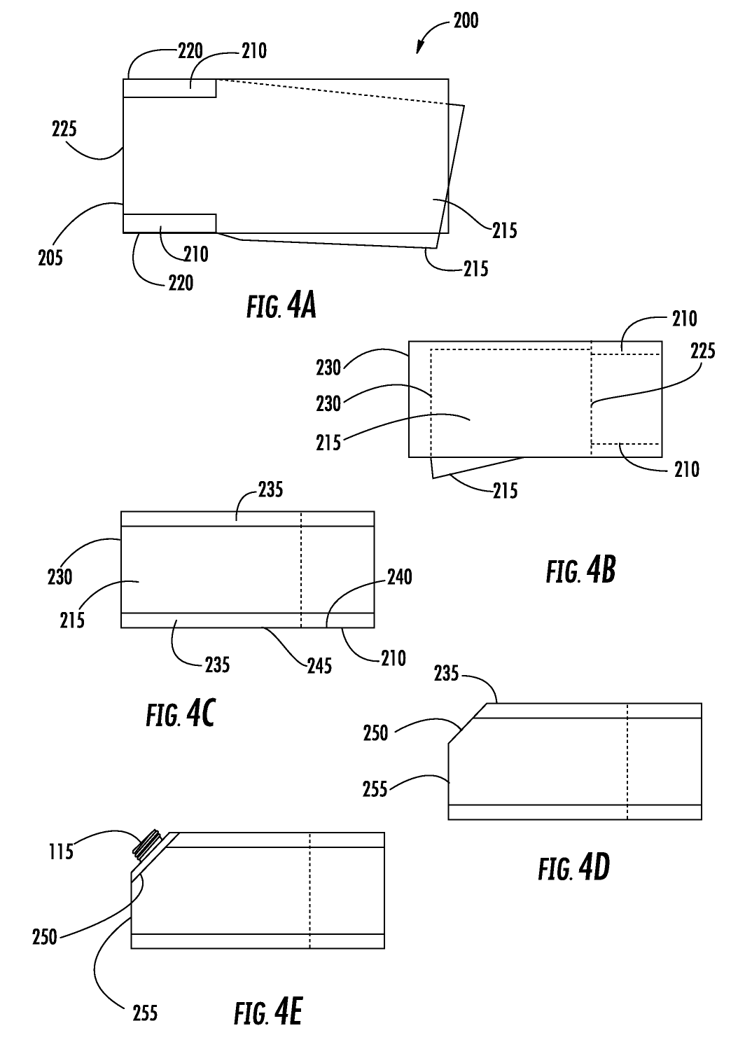

### DETAILED DESCRIPTION OF THE INVENTION

The inventor has determined that a number of problems exist with the current method of providing the colonoscopy prep material in a jug. First, the volume occupied by the jug is approximately four liters yet this jug is supplied to the pharmacist with less than 300 grams of solid power within the jug, which leaves the vast majority of the jug empty. As a consequence the transport of the jug is inefficient with the majority of the load transported being air within the almost empty jug.

Second, the inventor has also determined that individuals often have difficulties in preparing the mixture. For example, the individual may add insufficient quantities of water with the result being a lumpy mass that has problems dissolving in the water. This problem in reconstitution may be addressed by adding a first quantity of water sufficient to dissolve the entire power mixture and then adding a second quantity of water to result in four liters of fluid. Markings on the container in which the powder is reconstituted may provide sufficient guidance to accomplish this.

Third, the inventor has determined that individuals have difficulties in preparing the mixture because it is not possible to clearly determine whether or not the solid powder has sufficiently dissolved in the fluid. The walls of the reconstitution jug are relatively opaque such that it is not possible to visually ensure that the solid powder is completely dissolved.

The inventor has determined that one means of addressing each of these problems with the current reconstitution jug is use of a clear reconstitution bag, also known as a Marsupial Pouch™. Referring to FIG. 3, a reconstitution bag 100 includes two side walls 105, a spout section 110 having a threaded spout 115, a cap 120, a handle section 125 having one or more openings 130, and a base 135. The two side walls 105 are sealed together by a seam 140 that extends around the perimeter of the bag.

The side walls 105 are made from a clear, pharmaceutically acceptable polymer such as, for example, high density polyethylene, that is free of an opacifying agent or colorant. In particular, if the polyethylene polymer contains an opacifying agent or colorant, the amount of the opacifying agent or colorant should be present in an amount that is less than the amount that causes enough opacity such that it is difficult to determine whether or not a mixture of polyethylene glycol powder and water has sufficiently dissolved the powder. In one embodiment, one side wall may be free of an opacifying agent or colorant and the opposite side wall may include an opacifying agent or colorant. In this manner, the individual will have the ability to look through a side wall and determine that the solid material has sufficiently dissolved in the fluid.

The material making up side walls 105 and the rest of the reconstitution bag may be formed of a single layer or a multiple layer polymeric film. Also, in principle, any such film grade polymeric resin or material as generally known in the art of packaging can be employed. Preferably, a multilayer polymeric film structure is to be employed. Typically the multilayer polymeric sheet will involve at least three categorical layers including, but not limited to, an outermost structural layer, an inner barrier layer, and an innermost layer making contact with and compatible with the intended contents of the bag and capable of forming the necessary seals (e.g. most preferably heat-sealable) to itself. Other layers may also be present to serve as adhesive or “tie” layers to help bond these layers together.

The outermost structural layer is typically oriented polyester or oriented polypropylene, but can also include oriented polyamide (nylon) or paper or foil. This layer preferably is advantageously unaffected by the sealing temperatures used to make the package, since the package is sealed through the entire thickness of the multilayer structure. The thickness of this layer is typically selected to control the stiffness of the pouch, and may range from about 1 micron to about 100 microns, more preferably between about 5 to about 50 microns, and most preferably from about 8 to about 40 microns. Preferably, a BON (biaxially oriented nylon) film or any other kind of film that imparts flexibility and strong tear characteristics to the film is used for this layer.

The inner barrier layer(s) and the innermost layer which makes contact with the contents of the bag is preferably a polyethylene film which may be either high density or low density, having a thickness of between about 10 to about 500 microns, more preferably between about 20 to about 300 microns and most preferably between about 50 and about 200 microns. In one particular embodiment, a HDPE and a LDPE are employed together, with the HDPE having a thickness of between 50 and 200 microns, and the LDPE having a thickness of about 10 to about 50 microns.

An additional primer layer may be used in conjunction with the multilayer structure as is known in the art. The total thickness of the material making up side walls 105 and the remainder of the bag may have a thickness of about 10 microns to about 1,000 microns.

The threaded spout 115 may be made from a pharmaceutically acceptable polymer such as, for example, high density polyethylene. The polymer used in the spout may be free of an opacifying agent or a colorant, or both. The avoidance of an opacifying agent or colorant in the threaded spout is less important than in the side walls 105 because there is no reason to view through the spout.

An inside or outside portion of the spout preferably includes a seal 150 to prevent tampering with the interior contents as well as to prevent the introduction of humidity and bacteria to the interior contents of the bag 100. The seal is preferably a foil seal and is made up of one or more layers which are adhered to each other. For example, the seal may be made up of a laminated material consisting of a layer of pulp which is wax mounted to a layer of aluminum foil. The aluminum foil may then be heated to a temperature in the range of about 150-300 degrees Farenheit resulting in melting of the laminating wax between the pulp and foil.

Alternatively, one or more layers of the seal consist of aluminium foil bonded to a thermoplastic heat seal layer such as a layer of HDPE or LDPE (high-density or low-density polyethylene), the thermoplastic heat seal layer being sealed to the opening of the spout by its melting caused by inductive heating of the aluminium foil.

The heat seal layer and foil layer may have a thickness of about 0.001 mm to about 1 mm. The seal 150 may additionally include a PET (polyethylene terephthalate) layer having a thickness of about 0.001 mm to about 1 mm. A backing layer and wax layer may also be included, having a combined thickness of about 0.001 mm to about 3 mm.

The cap 120 may be made from a pharmaceutically acceptable polymer such as, for example, polypropylene. The polymer used in the cap may be free of an opacifying agent or a colorant, or both. The avoidance of an opacifying agent or colorant in the cap is less important than in the side walls 105 because there is no reason to view through the cap.

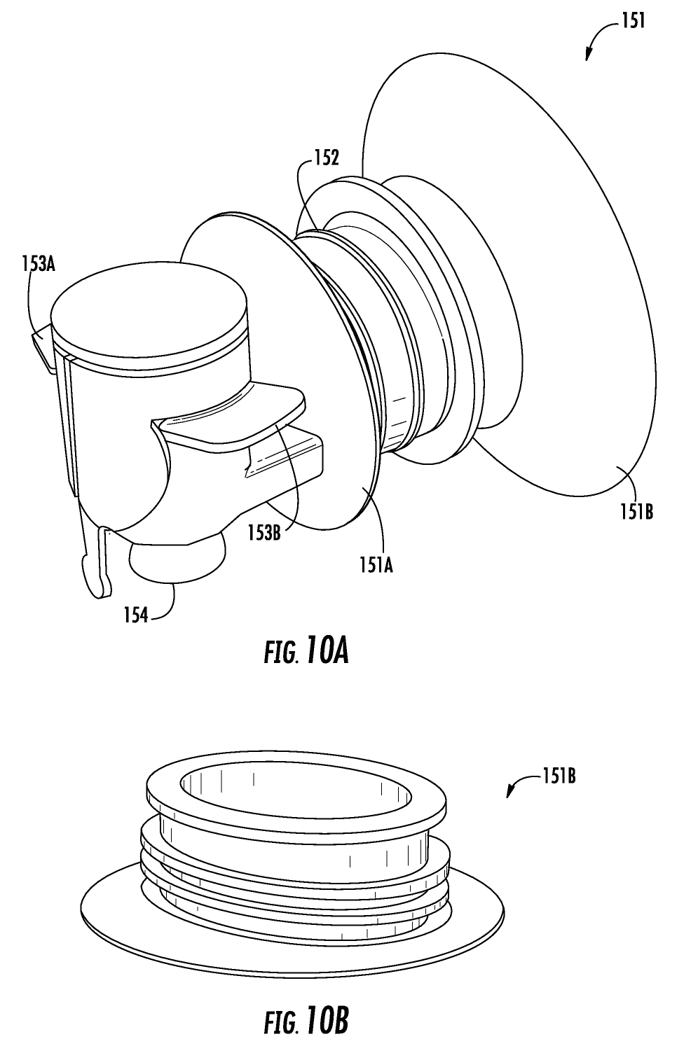

A spout fitment (not shown) may be used in conjunction with the spout 115 or spout 154 shown in FIG. 10A and discussed in detail below, which serves to seal the opening of the spout prior to use. The spout fitment is preferably made of high density polyethylene (HDPE) such as that having the trade name Alathon® H5618 manufactured by Lyondell Basell. The spout fitment can also be made of other types of HDPE and/or any other appropriate synthetic material, preferably polypropylene (PP) or polyethylene (PE), including low-density polyethylene (LDPE), medium-density polyethylene (MDPE) and high-density polyethylene (HDPE).

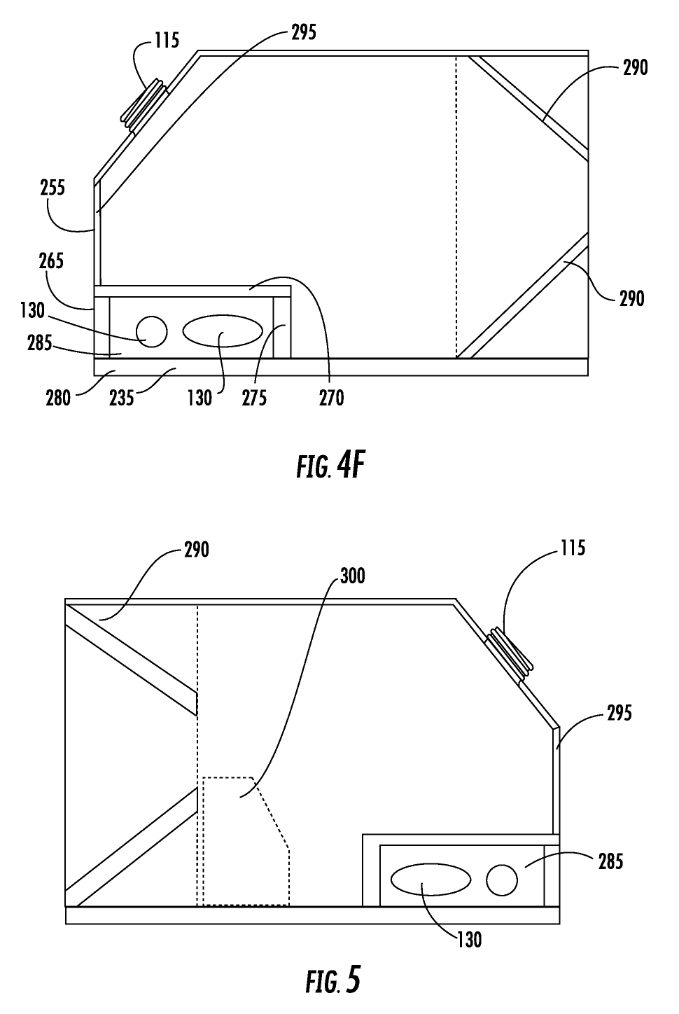

Referring to FIGS. 4a-f, in one embodiment of the configuration of the bag 100, the bag 100 may be made by taking a single, rectangular sheet of high density polyethylene (HDPE) or the multilayer film above-described 200 and folding the sheet across the width along the midpoint 205 of the length. The opposite edges of the sheet then are sealed for a length of approximately one to three inches 210 such that the resulting configuration is two sheets 215 joined for a short length 210 along both sides forming two edges 220 and a third edge 225 joining the two sealed edges and formed by the fold 205 of the two sheets 215. Referring to FIG. 4b, the free edges 230 of the two oppositely placed sheets 215 then are folded back over the sealed edges. Referring to FIG. 4c, the two sheets 215 next are sealed 235 along the length of both sides of the sheet, including over the two sealed edges. As a consequence of this sealing action, the seals along each edge have a first section 240 that has four layers sealed together and a second section 245 with only two layers sealed together. The first section forms the base 135.

Next, the threaded spout 115 is sealed between a section of the remaining open end between the two opposite sheets. The sealing operation may result in sealed sections of the end on opposite sides of the spout. Referring to FIG. 4d, in one embodiment, a portion of the sheets between one side and the open end may be made such that an angled section 250 connects the sealed side 235 and the open end 255. Referring to FIG. 4e, the threaded spout 115 may then be sealed into the angled section 250. After these sealing operations, the end adjacent to the spout remains open 255.

Referring to FIG. 4f, as a last step, one or more holding openings 130 are placed through the two sheets 215 in a position adjacent to the open end 255 but opposite from the threaded spout 115. First, three seals 265, 270, 275 are made between the two sheets such that the result is a square or rectangle with the seal 235 along the edge being the fourth edge 280. The four seals result in a fluid-tight section 285 of the bag through which fluid cannot flow. Two holes then are cut through the two sheets in the fluid-tight section. The two holes can be round, oblong, oval, etc. for decorative or ornamental purposes or for permitting a person to put fingers through the openings to grasp the bag.

Referring also to FIG. 4f, a pair of optional seals 290 may be made on each sheet to permit the base 135 to form on its on as and when the bag is full of fluid. The optional seals may be formed in one, two or four sealing operations.

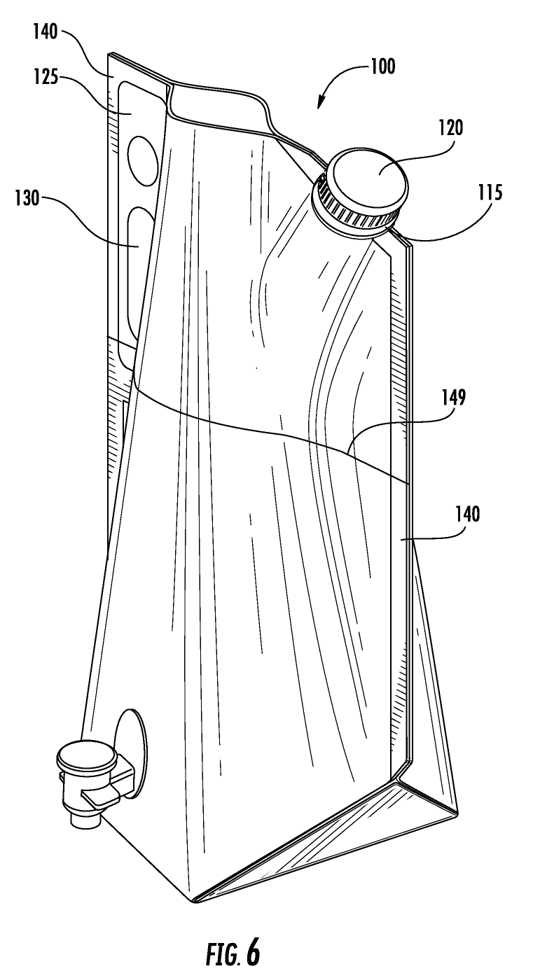

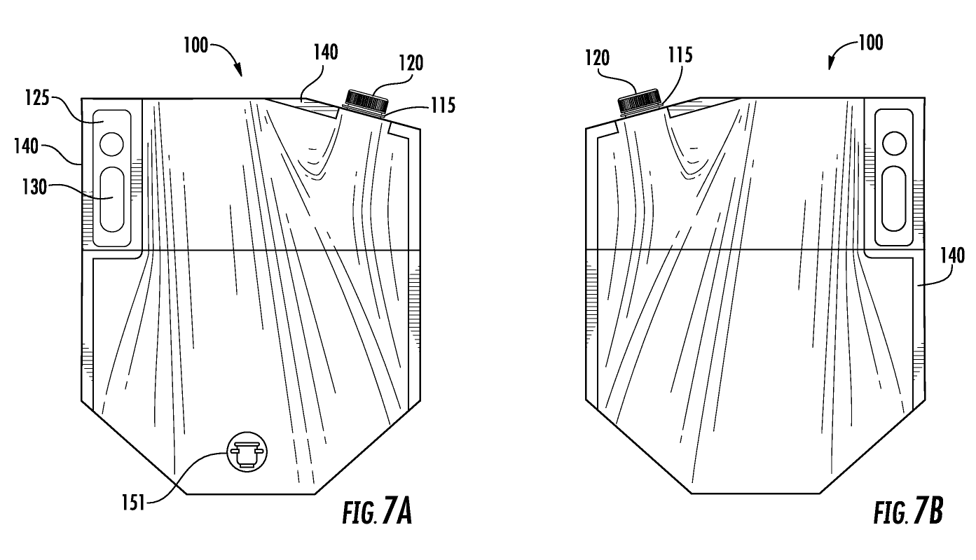

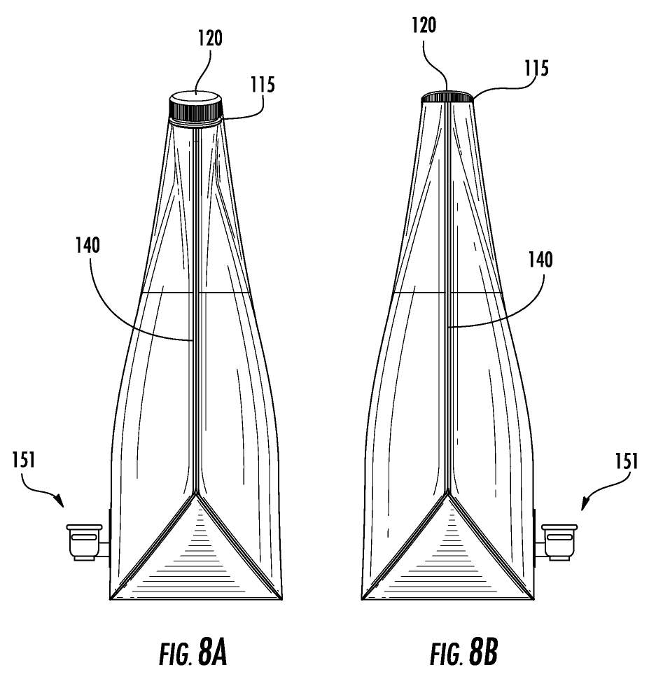

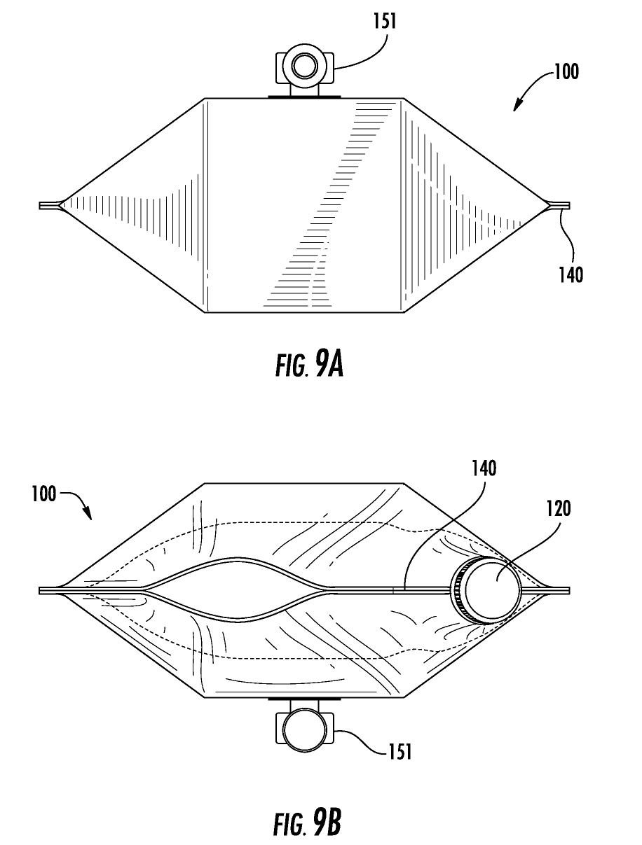

The reconstitution bag 100 may additionally include a tap and gland 151 near the base of the bag to aid in dispensing of the suspension which must be consumed by a patient. The tap and gland 151 is shown in FIG. 6 and in more detail in FIGS. 10A and 10B. The tap and gland 151 includes a dispensing valve and a tap body 151A having a male annular fitting which is inserted into a female gland 151B prior to use. The gland is preferably secured to one of the side walls 105 as is shown in FIG. 6 and may be generally circular in shape. A portion of the tap body 151A of the tap may be friction fit or lock fit within the gland or may alternatively be mated with the female portion of the gland by a threaded fit. Threading 152 is shown in FIG. 10A on the end of the tap body. A flexible diaphram may further be used in the connection of 151A and 151B in order to achieve a water tight seal. The diaphram may be made from a thermoplastic elastomer (TPE), for example a copolyester elastomer such as Arnitel® EM 400. FIG. 10A shows tap wings 153A and 153B which are in connection with a valve and may either be pressed down or turned radially to open the valve and allow the inside contents of the pouch to be dispensed through spout 154.

The tap body including wings is preferably integrally molded from a thermoplastic resin such as polyethylene or polypropylene, but can be molded from numerous materials such as rigid polyurethane, acetal, polyphenylene oxide, polyester, polyamide, polyphenylene sulphide, polyethylene terephthalate, ABS, polycarbonate, and polysulphone. Numerous criteria are considered when choosing a polymer such as cost, ease of molding, oxygen permeability, flexibility, strength, chemical resistance, and operational temperature. Polyolefins such as polypropylene and polyethylene are commonly used for similar types of single-method dispensing taps. It is of particular interest that a material be chosen for its structural behavior at cooler temperatures due to the recommendation to cool down the bowel prep composition prior to administration. Of particular preference for the tap body is a polypropylene/ethylene heterophasic copolymer. Of particular preference for the wings 153A and 153B is polyethylene resin such as that which is manufactured by Safripol and given the trade name Safrene® D 7255.

The gland is typically made from LDPE or HDPE and may be injection molded, blow molded, extruded or the like. Of particular use is linear low-density polyethylene (LLDPE) such as that which is manufactured by SABIC® including SABIC® LLDPE M200024 00900.

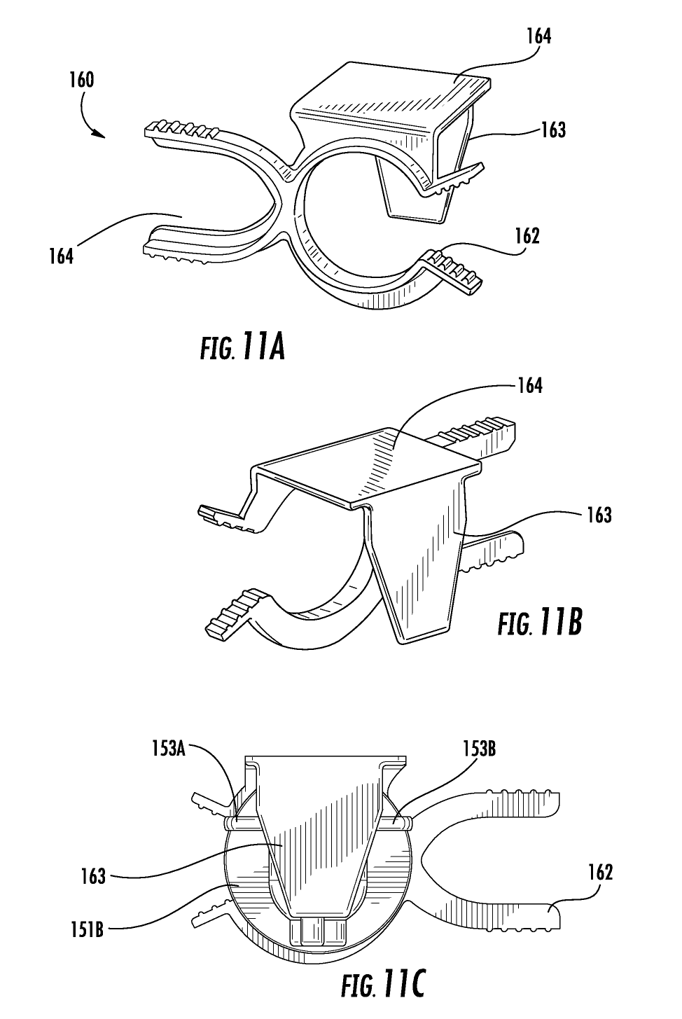

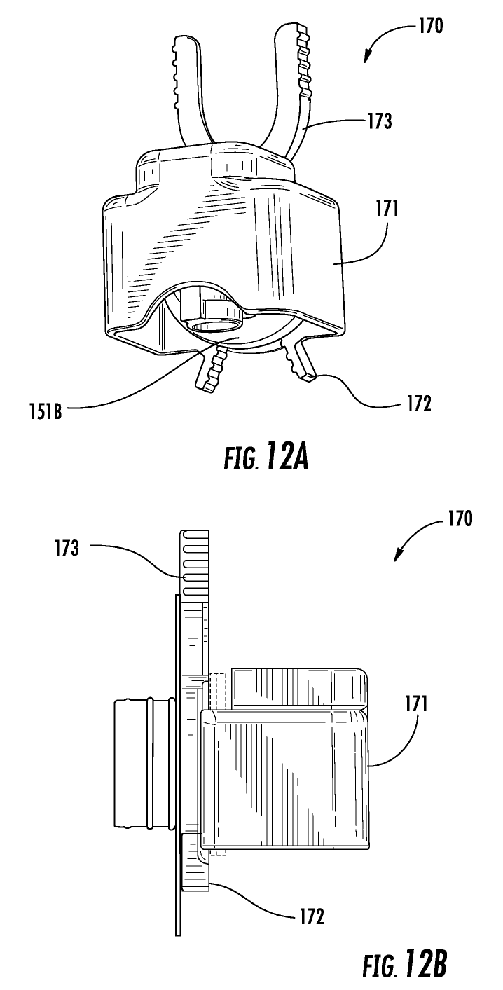

FIGS. 11A, 11B, 11C, 12A and 12B depict a child restraint cap which may be used in conjunction with tap and gland 151 in order to prevent a child from accessing the wings 153A and 153B of the tap body 151A. A first embodiment of the child restraint cap 160 is provided in FIGS. 11A and 11B, which includes V portion 161, scissor portion 162 which has a generally circular shape to fit around circular gland 151B, blocking plate 163 and top plate 164. These portions are all preferably part of a single unitary piece which is sized and shaped such that the scissor portion 162 is secured around the generally circular gland 151B such that top plate 164 blocks access to the wings 153A and 153B from above the wings and blocking plate 163 blocks access to the wings from the front of the wings. The shape of the top plate and the blocking plate may vary and is chosen based on minimizing the amount of material needed while also providing a shape and size which is capable of covering the wings. The child restraint cap 160 may be removed from the bag by applying an inward pressure to both ends of V portion 161 which allows the diameter of scissor portion to increase such that the cap 160 may be slid off the gland 151B.

FIGS. 12A and 12B depict a second embodiment of the child restraint cap 170 including body 171 being substantially cube shaped with rounded corners, scissor portion 172 and V shaped portion 173. The cube shaped body 171 has top, left, right, front and back wall and preferably no bottom wall to permit the end of the spout 154 to vary in size and to extend beyond the length of the walls of body 171. The back wall of the body 171 preferably includes a substantially circular cut-out to accommodate the shape of the gland 151B such that the cut-out substantially surrounds the perimeter of the gland to form a tight and secure friction fit. At any point along the circumference of the circular cut out there extends a scissor portion 172 and V shaped portion 173. The child restraint cap 170 may be removed from the bag by applying an inward pressure to both ends of V portion 173 which allows the diameter of scissor portion to increase such that the cap 170 may be slid off the gland 151B.

The material used to form either child restraint caps 160 or 170 should be a durable yet flexible plastic due to the requirement constant compression of the V shaped portions 161 and 173, respectively. LDPE is one such preferred material.

The child restrain locks are considered to be a particularly important feature of the reconstitution bag of the present invention due to the potentially toxic nature of the contents placed within the bags and extreme circumstances that would result in having the pharmaceutical contents be ingested by an unauthorized/unprescribed user.

FIG. 6 also shows fill line 149 which is particularly useful to include in allowing the user to understand how much water to pour into the bag 100 without having to pre-measure any amount.

The bag 100 may come in kit form and may include a flavour packet and instructions for administration of the pharmaceutical composition, such as adding a fluid such as water to the bag up to the fill line, mixing or shaking the suspension together for a period of time or until the powder composition is dissolved, chilling the suspension, and/or drinking the suspension.

The resulting bag is suitable for efficient transport to a pharmaceutical manufacturer for filling with a solid, pharmaceutical powder. It is estimated that the empty bags with the spout in place form a volume that is approximately one eighth to one fifteenth of the volume of four liter jugs, one tenth to one fourteenth of the volume of four liter jugs, or one eleventh to one thirteenth of the volume of four liter jugs, or one twelfth of the volume of four liter jugs. This difference in empty volume provides significant savings in transportation and storage costs, along with the associated ecological benefits.

Referring to FIG. 5, once the bags are at the pharmaceutical manufacturer, the bags may be filled with a colonoscopic bowel preparation in solid powder form, such as a polyethylene glycol powder mixture 300. In one embodiment, the powder may be filled into the empty bag through the unsealed opening and then the opening 255 sealed with seal 295. The threaded spout may have the cap placed over the spout before or after the bag is filled. In another embodiment, the opening 255 may be sealed and the powder filled into the empty bag through the opening in the threaded spout. In this second embodiment, the cap is placed over the spout after the bag is filled. In both embodiments, a small pouch of a flavoring agent is attached to the outside surface of the bag, such as by tape or adhesive.

The filled bags then may be labelled with appropriate labels according to US Food and Drug Administration regulations for pharmaceutical products, packaged with any required instructions, and stored for shipment to pharmacists, pharmaceutical wholesalers and the like who distribute pharmaceutical products. Because of the reduced size of the filled reconstitution bags in comparison to the currently used reconstitution jugs, both the storage and transportation costs are advantageously reduced.

When a pharmacist receives a prescription for the filled reconstitution bag, the pharmacist typically will add the flavoring agent to the polyethylene glycol mixture and provide the bag to the patient. The patient then may take the bag home and add a clear fluid to the bag through the threaded spout. The mixing instructions may vary. For example, in one embodiment a warm fluid is poured into the bag until the level of the fluid reaches a first fill mark. The cap then is threadably attached to the spout and the bag shaken until the powder mixture is dissolved. Because the side walls of the bag are clear, the user can easily see whether or not the powder is dissolved. The cap then is removed and the remaining volume of the bag filled with the fluid, although cooled rather than warm fluid is used. The bag may include a second fill mark that indicates when the desired total volume of fluid has been added. The cap then is placed on the spout, the filled bag shaken to mix the fluids, and the filled bag then is placed in refrigerated storage until administration.

In another embodiment, fluid is added to the bag through the open spout until the bag is full of fluid. The cap then is placed on the spout and the bag is shaken until the polyethylene powder is dissolved. The filled bag then is placed in refrigerated storage until administration.

The above steps are believed to address the three problems addressed above: a bag that occupies a reduced volume that provides for efficient storage and transportation, clear wall or walls of the bag to ensure dissolution of the polyethylene glycol powder mixture, and markings to provide guidance for adding sufficient quantities of fluid to the bag for mixing.

The patient preparing for the colonoscopy next follows the steps outlined above for using the currently approved jug of propylene glycol for colonoscopy prep. In these steps, the reconstitution bag of the invention may have a number of useful features. First, the openings in the bag may be used as a handle to hold the bag when mixing the ingredients to dissolve the powder and when pouring the fluid contents into a glass. Second, when the bag is full of fluid, the base will expand and form a surface such that the bag will sit upright with the spout at the top.

It should be understood that “administering” the composition can be understood to mean delivering or causing to be delivered the composition into the body of an individual. For example, “administering” can be understood to mean a healthcare professional prescribing, supervising, or managing the formal taking of the composition by an individual.

It should be understood that a number of variations of the bag are possible. First, while the fluid-tight section is illustrated as being formed in the shape of a rectangle or square, other shapes are possible. In one alternative embodiment, an edge of the section is at an angle to the outer edge of the bag. Because of the angled seals, there is the reduced likelihood of pockets being formed in the bag that may hold undissolved polyethylene glycol powder.

Second, the openings in the fluid-tight section may be round, oblong, oval, square, etc. or a combination of two shapes. Further, the area of each opening may be the same or different.

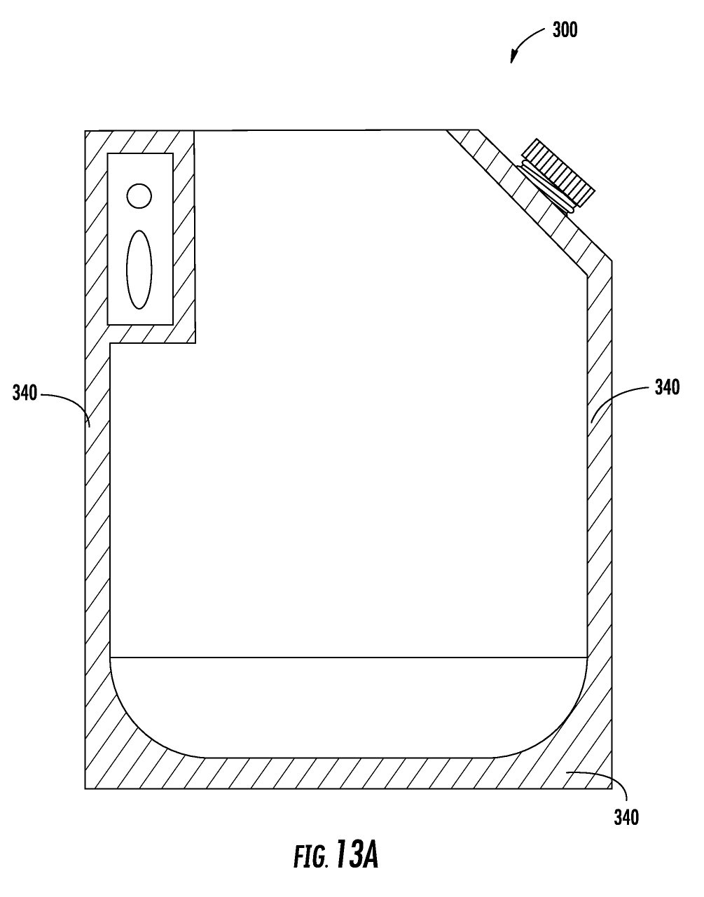

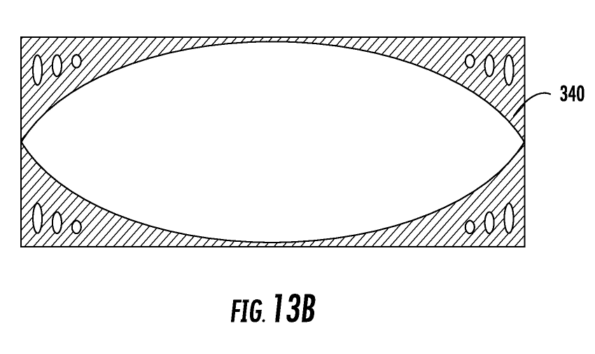

Varying seal shapes may be used in conjunction with the base of the bag to allow the bag to remain upright when full of fluid. For example, FIGS. 13A and 13B provide a front view and a bottom view of a reconstitution bag 300 having an alternative seal shape to form a different shape and size of base 335 than that which is shown in FIG. 3. For example, FIG. 13A shows seal 340 to be in a generally semi-circle shape at the bottom portion of the reconstitution bag 300, which allows the base 335 of the bag to take on a generally elliptical shape.

What is claimed is:

1. A pharmaceutical composition comprising a colonoscopic bowel preparation in solid powder form contained within a polymer bag having an opening.

2. The pharmaceutical composition of claim 1, wherein the pharmaceutical composition comprises polyethylene glycol 3350, potassium chloride, sodium bicarbonate sodium chloride, and, optionally, sodium sulfate anhydrous.

3. The pharmaceutical composition of claim 2, wherein the pharmaceutical composition comprises about 200 to 250 grams of polyethylene glycol 3350, 2.5 to 3.5 grams of potassium chloride, 6.5 to 7.5 grams of sodium bicarbonate and 5.5 to 6.5 grams of sodium chloride.

4. The pharmaceutical composition of claim 3, wherein the pharmaceutical composition is comprises about 236 grams of polyethylene glycol 3350, about 2.97 grams of potassium chloride, about 6.74 grams of sodium bicarbonate and about 5.86 grams of sodium chloride.

5. The pharmaceutical composition of claim 1, wherein the polymer bag comprises high density polyethylene.

6. The pharmaceutical composition of claim 1, wherein the polymer bag has a volume of approximately 4 liters when filled with fluid.

7. The pharmaceutical composition of claim 1, wherein the polymer bag includes at least one opening through the bag, the opening in the bag being defined by a continuous edge of the polymer bag forming a seal between an interior volume of the bag and the opening through the bag.

8. The pharmaceutical composition of claim 1, wherein the opening comprises a threaded spout and a cap threadably mountable to the spout, wherein the threaded spout forms a passageway to an interior volume of the bag.

9. The pharmaceutical composition of claim 1, wherein the bag contains a base.

10. The pharmaceutical composition of claim 1, wherein the polymer bag is a clear polymer material.

11. The pharmaceutical composition of claim 1, wherein the polymer bag includes one or more fill markings on the bag.

12. A method for administering a pharmaceutical composition comprising a colonoscopic bowel preparation in solid powder form, the method comprising:
obtaining a polymer bag having a threaded spout and a cap threadably mountable to the spout, wherein the threaded spout forms a passageway to an interior volume of the bag, the pharmaceutical composition in the bag comprising a solid powder; and
adding a fluid to the bag.

13. The method of claim 12, further comprising mixing the solid powder with the fluid to dissolve the solid powder.

14. The method of claim 12, wherein approximately four liters of fluid are added to the bag.

15. The method of claim 13, wherein a first portion of the fluid is added to the bag until a mark on a surface of the bag is reached, the bag is shaken to mix the pharmaceutical composition with the fluid, and then a second portion of the fluid is added to the bag.

16. The method of claim 12, wherein the pharmaceutical composition comprises polyethylene glycol 3350, potassium chloride, sodium bicarbonate, sodium chloride and, optionally, sodium sulfate anhydrous.

17. The method of claim 16, wherein the pharmaceutical composition comprises about 200 to 250 grams of polyethylene glycol 3350, 2.5 to 3.5 grams of potassium chloride, 6.5 to 7.5 grams of sodium bicarbonate and 5.5 to 6.5 grams of sodium chloride.

18. The method of claim 12, wherein the polymer bag is a clear polymer material.

19. The method of claim 12, wherein the polymer bag comprises high density polyethylene.

20. A kit for administering a pharmaceutical composition comprising a colonoscopic bowel preparation in solid powder form, the kit comprising:
a polymer bag having a threaded spout and a cap threadably mountable to the spout, wherein the threaded spout forms a passageway to an interior volume of the bag;
an optional flavour packet; and
instructions for administering the pharmaceutical composition comprising adding a fluid to the bag.
